(19) United States
(12) Patent Application Publication
(10) Pub. No.: US 2025/0256056 A1
(43) Pub. Date: Aug. 14, 2025

(54) METHODS, SYSTEMS, AND DEVICES FOR THE TREATMENT OF STRESS DISORDERS

(71) Applicant: Vivos Therapeutics, Inc., Littleton, CO (US)

(72) Inventor: Scott A. Simonetti, East Islip, NY (US)

(73) Assignee: Vivos Therapeutics, Inc., Littleton, CO (US)

(21) Appl. No.: 19/048,897

(22) Filed: Feb. 8, 2025

Related U.S. Application Data
(60) Provisional application No. 63/551,842, filed on Feb. 9, 2024.

(51) Int. Cl. A61M 21/02; A63B 71/08

(52) U.S. Cl. CPC A61M 21/02; A63B 71/085; A61M 2207/00

(57) ABSTRACT

Providing a safe oral orthotic appliance for treatment of stress disorders and stress disorder symptoms. The oral orthotic reduces overactive muscles, headaches, and rhinosinusitis while decreasing stress and improving sleep without exasperating respiratory disorders.

### CROSS-REFERENCE TO RELATED APPLICATIONS

This application claims priority to, and the benefit of U.S. Provisional Application No. 63/551,842, filed Feb. 9, 2024, and titled “METHODS, SYSTEMS, AND DEVICES FOR THE TREATMENT OF STRESS DISORDERS,” which is incorporated by reference herein in its entirety for all purposes.

### FIELD

The present disclosure relates to treatment of bruxism, TMD, and stress disorders by reducing activation of the sympathetic nervous system, among other things.

### BACKGROUND

Bruxism is excessive teeth grinding or jaw clenching and can lead to headaches, jaw and facial pain, mild to severe tooth wear, tooth fractures, receding gums, and impaired sleep. The prevalence of bruxism is reported to be between 8% and 31% of the adult population. People grind their teeth during the day as well as during their sleep which could lead to Temporomandibular Joint Dysfunction (TMD). Bruxism may be associated with emotional factors such as anxiety or post-traumatic stress disorder, resulting in compromised function.

Heart Rate Variability (HRV) involves the heart's rhythms and is characterized by both complexity and stability over longer time scales that reflect both physiological and psychological functional status of these internal self-regulatory systems. HRV can be viewed as a measure of self-regulatory capacity. When HRV is low, the BioRegulatory homeostasis is lost and resiliency is decreased. Over time, low HRV will allow dysregulation to become the normal, as the disease process ensues. Strong emerging evidence supports the connection between low HRV and migraine and tension like headaches. By improving HRV and preventing a worsening of sleep quality, TMJ symptoms may be improved or avoided. Researchers also found that subjective sleep quality worsened progressively before the onset of painful TMD. By restoring the autoregulatory capacity and enabling a flexible autonomic nervous system (ANS), the pain syndromes associated with sympathetic hyperactivity, parasympathetic underactivity, and low HRV can be reduced. TMD predictors have been carefully analyzed (Orofacial Pain: Prospective Evaluation and Risk Assessment (OPPERA) study)—relative to controls, TMD cases displayed a dysfunction in autonomic activity characterized by reduced HRV at rest and in response to physical and physiological stressors. Migraine and chronic pain patients were found to have reduced parasympathetic activity and sympathetic predominance and lower Vagal mediated HRV.

Emerging evidence indicates that dysregulation of the autonomic nervous system contributes to the onset and persistence of Temporomandibular Joint (TMJ) dysfunction and related conditions. HRV measures the balance between the sympathetic and parasympathetic autonomic nervous systems. HRV is considered a window into Autonomic Nervous System Function, and low HRV has been shown to be associated with many stress and pain syndromes. HRV is a well-known biomarker that can be used to evaluate sympathetic/parasympathetic activity in patients with neurological disorders because it can be determined simply, accurately and noninvasively. High HRV is healthy and represents resiliency, autonomic balance and coherence, while low HRV indicates autonomic dysfunction. Reduced parasympathetic activity with sympathetic predominance has been found in migraine patients. There is an association between sympathetic nervous system activation in migraine and tension-type headache sufferers and also between autonomic nervous system factors and TMD such as reduced HRV and elevated heart rate. In addition, there is an association between emotional factors such as anxiety and bruxism, resulting in compromised masticatory function. The anxiety from the pain creates a stressor that creates masticatory muscle hyperactivity which is the most frequent mechanism influencing myofascial pain. This leads to a vicious cycle of chronic TMJ/facial pain and headaches that will concurrently be seen with other central sensitization syndromes that all present with reduced HRV.

Conventional methods of treating bruxism or TMD include flat-plane splints and maxillary bite guards. Conventional methods are associated with aggravation of respiratory disturbances and can increase masseter muscle activity. A person with bruxism or TMD will often have an underlying breathing disorder, aggravated by conventional methods. Full coverage splints have no way to reduce maximum bite force and overactive masticatory muscles, which can lead to a long-term exacerbation of the original etiology of excessive muscle activity.

Therefore, improved methods for treatment of bruxism, TMD, and stress disorders are desirable.

### SUMMARY

According to various embodiments, treatments of stress disorders, bruxism, and TMD are disclosed. In various embodiments, the present disclosure provides for a method for making an oral orthotic. In various embodiments, the oral orthotic may be for treatment of a stress disorder. In various embodiments, the method may comprise conducting an initial inventory of stress disorder symptoms. In various embodiments, the stress disorder symptoms may include at least one of heart rate variability (HRV), bioregulatory homeostasis, frequency of headaches, intensity of headaches, frequency of anxiety, or intensity of anxiety. In various embodiments, the method may comprise providing the oral orthotic to a patient. In various embodiments, the oral orthotic may have a left portion, a right portion, a lingual portion, and a bite block. In various embodiments, the bite block may be configured and disposed exclusively on one of the left portion or the right portion to extend over a subset of teeth of a patient along the mandibular bone of the patient. In various embodiments, the method may comprise positioning the oral orthotic in a mouth of the patient. In various embodiments, the method may comprise visually inspecting a fit of the oral orthotic. In various embodiments, the method may comprise evaluating contact points of an upper portion of the bite block with at least one maxillary tooth of the patient. In various embodiments, the method may comprise evaluating a closed lip posture of the patient with the oral orthotic. In various embodiments, the method may comprise evaluating a tongue posture of the patient with the oral orthotic.

In various embodiments, the method may comprise determining a first period of time for wearing the oral orthotic based on the initial inventory. In various embodiments, the method may comprise determining a second period of time for removing the oral orthotic based on the initial inventory. In various embodiments, the method may comprise determining a third period of time for repeating the wearing and removing based on the initial inventory.

In various embodiments, the method may comprise evaluating of the tongue posture is conducted while the patient conducts the series of breaths.

In various embodiments, the method may comprise removing the bite block from the mouth of the patient and adjusting the bite block based on the evaluation of the tongue posture.

In various embodiments, the adjusting may include at least one of removing material from the bite block or adding material to the bite block.

In various embodiments, the method may comprise visually inspecting mentalis strain of the patient during the breathing and wherein the adjusting is further based on the mentalis strain.

In various embodiments, the method may comprise repositioning the orthotic in the mouth of the patient after the adjusting. In various embodiments, the method may comprise reevaluating the tongue posture while the patient conducts a second series of breaths.

In various embodiments, the present disclosure provides for treatment of a stress disorder with an oral orthotic. In various embodiments, the method may comprise positioning the oral orthotic in a mouth of a patient. In various embodiments, the oral orthotic may have a left portion, a right portion, a lingual portion, and a bite block. In various embodiments, the bite block may be configured and disposed exclusively on one of the left portion or the right portion to extend over a subset of teeth of a patient along the mandibular bone of the patient.

In various embodiments, the method may comprise wearing the oral orthotic for the first predetermined period of time. In various embodiments, the method may comprise removing the oral orthotic for a second predetermined period of time. In various embodiments, the wearing includes contacting an upper portion of the bite block with at least one maxillary tooth of the patient. In various embodiments, the wearing includes closing the lips of the patient around the oral orthotic to a closed lip posture. In various embodiments, the wearing includes breathing a series of breaths while maintaining the contact and the closed lip posture.

In various embodiments, the method may comprise, in response to the wearing the oral orthotic, maintaining a proper tongue posture during the breathing.

In various embodiments, the method may comprise repeating the wearing and removing steps over a third predetermined period of time. In various embodiments, the first period of time, the second period of time, and the third period of time are based on at least one patient-specific symptom of the stress disorder.

In various embodiments, the present disclosure provides for a method for making an oral orthotic. In various embodiments, the method may comprise conducting an initial inventory of stress disorder symptoms. In various embodiments, the method may comprise creating a mold for the oral orthotic, the mold may have a left portion, a right portion, and a lingual portion. In various embodiments, the method may comprise determining an ideal side of the mouth of the patient for a bite block based on the initial inventory and the mold. In various embodiments, the ideal side corresponds either to the left portion or the right portion.

In various embodiments, the method may comprise providing the oral orthotic to a patient. In various embodiments, the oral orthotic may have a bite block configured and disposed exclusively on the ideal side to extend over a subset of teeth of a patient along the mandibular bone of the patient.

In various embodiments, the method may comprise positioning the oral orthotic in a mouth of the patient. In various embodiments, the method may comprise visually inspecting a fit of the oral orthotic. In various embodiments, the method may comprise evaluating contact points of an upper portion of the bite block with at least one maxillary tooth of the patient. In various embodiments, the method may comprise evaluating a closed lip posture of the patient with the oral orthotic. In various embodiments, the method may comprise evaluating a tongue posture of the patient with the oral orthotic.

In various embodiments, the method may comprise adjusting the bite block based on the tongue posture of the patient. In various embodiments, the method may comprise conducting a reevaluation inventory of the stress disorder symptoms.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the following detailed description and claims in connection with the following drawings. While the drawings illustrate various embodiments employing the principles described herein, the drawings do not limit the scope of the claims.

FIG. 1 illustrates a perspective view of an oral orthotic with mandibular splint utilizing a posterior unilateral contact, in accordance with various embodiments;

FIG. 2 illustrates a top plan view of an oral orthotic positioned along a mandible, in accordance with various embodiments;

FIG. 3 illustrates a front plan view of an oral orthotic positioned along the mandible of FIG. 1, in accordance with various embodiments;

FIG. 4 illustrates a rear plan view of an oral orthotic positioned along the mandible of FIG. 1, in accordance with various embodiments;

FIG. 5 illustrates a cross-sectional view of an oral orthotic positioned along the mandible, taken along section line 5-5 of FIG. 2, in accordance with various embodiments;

FIG. 6 illustrates an exploded perspective view of an oral orthotic and mandible, in accordance with various embodiments;

FIG. 7 illustrates a top plan view of an oral orthotic of FIG. 1, in accordance with various embodiments;

FIG. 8 illustrates a method of making an oral orthotic for treating a stress disorder, in accordance with various embodiments;

FIG. 9 illustrates a method of using an oral orthotic for treating a stress disorder, in accordance with various embodiments;

FIG. 10 illustrates the relationship between homeostasis and auto regulatory capacity with multiple allostatic loads; and

FIGS. 11A-11D illustrate various proper and improper tongue postures, in accordance with various embodiments.

FIG. 12 illustrates a method of making an oral orthotic for treating a stress disorder, in accordance with various embodiments.

### DETAILED DESCRIPTION

The following detailed description of various embodiments herein makes reference to the accompanying drawings, which show various embodiments by way of illustration. While these various embodiments are described in sufficient detail to enable those skilled in the art to practice the disclosure, it should be understood that other embodiments may be realized and that changes may be made without departing from the scope of the disclosure. Thus, the detailed description herein is presented for purposes of illustration only and not of limitation. Furthermore, any reference to “singular” includes plural embodiments, and any reference to “more than one” component or step may include a singular embodiment or step. Also, any reference to “attached,” “fixed,” “connected,” or the like may include permanent, removable, temporary, partial, full or any other possible attachment option. Additionally, any reference to “without contact” (or similar phrases) may also include reduced contact or minimal contact. It should also be understood that unless specifically stated otherwise, references to “a,” “an,” or “the” may include one or more than one and that reference to an item in the singular may also include the item in the plural. Further, all ranges may include upper and lower values and all ranges and ratio limits disclosed herein may be combined.

For the sake of brevity, conventional approaches for taking a mold, forming an oral orthotic, adding material to an oral orthotic, removing material from an oral orthotic and/or the like may not be described in detail herein. Furthermore, the connecting lines shown in various figures contained herein are intended to represent exemplary functional relationships and/or physical or communicative couplings between various elements. It should be noted that many alternative or additional functional relationships or physical connections may be present in a practical system and/or related methods of use, for example a method of treating post traumatic stress disorder, depression, anxiety, or parasympathetic nervous system disorders.

As used herein, the term “clinician” refers to a doctor, nurse, or other care provider and may include support personnel. The term “patient” refers to an individual being treated by a clinician. The term “distal” refers to the portion of the component being described which is further from a clinician, while the term “proximal” refers to the portion of the component being described which is closer to the clinician. The term “left” refers to the left portion of an anatomical structure, such as a mandible, as viewed by a clinician. The term “right” refers to the right portion of an anatomical structure as viewed by the clinician. The term “parafunctional” is defined herein to refer to habits such as bruxism (e.g., clenching of the teeth or jaw), and other habits which are unrelated to eating or speaking.

Conventional methods of treating bruxism or TMD include flat-plane splints and maxillary bite guards. Conventional methods are associated with aggravation of respiratory disturbances and can increase masseter muscle activity. This is because the preferred placement for the tongue, resting against the roof of the mouth behind the front teeth, may be altered by maxillary splints or dual splints with both mandibular and maxillary guards. Maxillary splints or guards may trigger the body to remove the tongue from proper tongue posture, resulting in a greater occlusion or interruption of the person's airway. A person with bruxism or TMD will often have an underlying breathing disorder, which is then aggravated by conventional methods. Full coverage splints have no way to reduce maximum bite force and overactive masticatory muscles, which can lead to a long-term exacerbation of the original etiology of excessive muscle activity.

The oral orthotic of the present disclosure provides for treatment of bruxism, TMD, and various stress disorders without aggravating underlying respiratory disturbances or increasing masseter muscle activity. Rather, the oral orthotic of the present disclosure provides a mandibular splint utilizing a posterior unilateral contact to trigger a reduction of an overactive stress response and improving autonomic nervous system (ANS) function. The mandibular splint, as opposed to a maxillary splint or a dual splint with both mandibular and maxillary guards, encourages proper tongue posture. As a result, the mandibular splint (also called an orthotic or guard herein) may improve respiratory function over conventional treatment methods and does not aggravate underlying respiratory disturbances. The mandibular splint may also restore diaphragmatic breathing. Improved respiratory function and diaphragmatic breathing both support de-activation of the sympathetic nervous system and activation of the parasympathetic nervous system. Increased parasympathetic nervous system function is associated with decreased stress and stress disorders. Further, a posterior unilateral contact, as opposed to a bilateral contact, limits contact between the mandibular and maxillary teeth. A unilateral contact signals the buccolabial muscles, facial muscles, or muscles associated with the TMJ to reduce activation or stop activation. This can be especially important during subconscious responses to stress or while sleeping. As a result, the user's buccolabial or facial muscles spend less time activated, which leads to improvement in bruxism, TMD, headaches, etc.

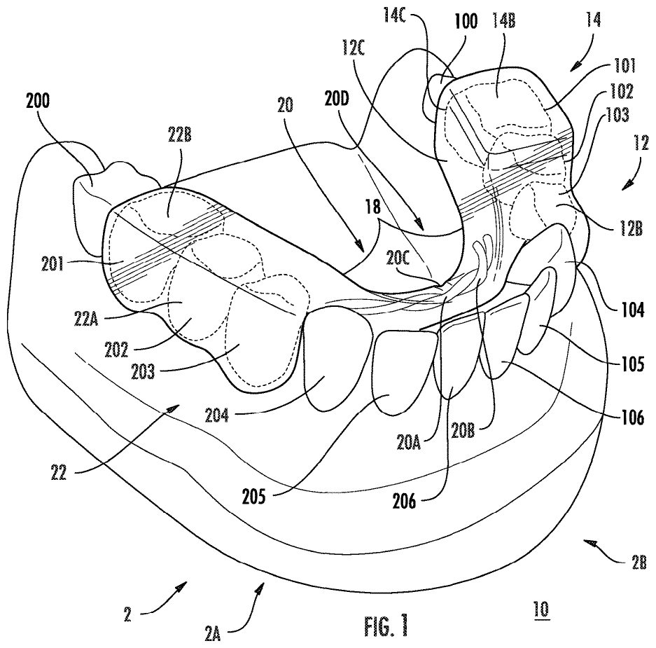

With initial reference to FIG. 1, an oral orthotic 10 is illustrated, in accordance with various embodiments. For purposes of clarity, the orthotic 10 will be described as associated with the mandible of a patient, however the orthotic 10 may likewise be adapted to be positioned along the maxilla of the patient. The orthotic 10 includes a left portion 22, a lingual portion 20, and a right portion 12. An occlusal plane 4 is defined by teeth attached to the upper surface of a mandible 2, further defining a left occlusal plane 4A and a right occlusal plane 4B. The lingual portion 20 is dimensioned to contour an arch 18 defined by the mandible. When inserted into the patient's mouth, the orthotic 10 attaches and is positioned adjacent to the mandible 2.

The left portion 22 includes an outer portion 22A, an upper portion 22B, and an inner portion 22C. The outer portion 22A, upper portion 22B, and inner portion 22C define a left arch which is dimensioned to align with contours of teeth located along the left portion 2A of a patient's mouth. The left arch extends along the upper surface of the left occlusal plane 4A between a left first molar 201 and a left first bicuspid 203. It is contemplated that in embodiments, the left arch may extend between a left cuspid 204 and a left second molar 200 or varying positions therebetween. The upper portion 22B and the inner portion 22C connect to a left portion of the lingual portion 20.

The lingual portion 20 extends along the arch defined by the mandible 2 between the left first bicuspid and a second portion of the right first bicuspid. The lingual portion 20 includes lingual arch 18 which encloses a first wire 20A and a second wire 20B. The first and second wire 20A, 20B are braided in a helical fashion to support the lingual arch 18, thereby applying forces in opposite directions when forces are received by either the left portion 22 or the right portion 12 of the orthotic 10. The first and second wire 20A, 20B additionally apply counter forces to torsional forces applied to the orthotic 10 along either the left portion 22 or right portion 12. The lingual portion 20 connects to the right portion 12 of the orthotic 10. The first and second wire 20A, 20B are constructed of stainless steel. In embodiments the first and second wire 20A, 20B is constructed of a material with greater rigidity than the material selected to enclose the first and second wire 20A, 20B. It is further contemplated that, in embodiments a resilient member e.g., may be located in place of the first and second wire 20A, 20B.

The lingual portion 20 further defines a lingual guide plane 20D. The lingual guide plane 20D is offset from the anterior portion of the teeth located along the arch 18 of the patient's mandible. As a result, the lingual portion 20 does not come into contact with the anterior portion of the patient's teeth, thereby reducing the potential to reposition the right and left cuspids (204, 104), lateral incisors (205, 105), and central incisors (206, 106). Additionally, due to the offset between the lingual portion 20 and the anterior portion of the patient's teeth, the chance of gingival irritation is also reduced. The lingual guide plane 20D redirects the patient's tongue when the tongue is advanced toward the lingual portion 20.

The lingual guide plane 20D further defines an indent 20C located along the lingual portion 20. More particularly, the indent 20C is located along the lingual portion 20 between the central incisors (206, 106) of the patient when the orthotic 10 is located in the patient's mouth. The indent 20C is configured to receive a lingual frenulum of the patient when the orthotic 10 is located in the patient's mouth.

The right portion 12 includes an outer portion 12A, an upper portion 12B, and an inner portion 12C. The outer portion 12A, upper portion 12B and inner portion 12C define a right arch which is dimensioned to align with a contour of teeth located along the right portion 2B of the mandible 2. The right arch extends along an upper surface of the right occlusal plane 4B between a right first molar 101 and a portion of a right first bicuspid 103. It is contemplated that in embodiments, the right arch may extend between a right cuspid 104 and a right second molar 100, or varying positions therebetween. The upper portion 12B and the inner portion 12C connect to a right portion of the lingual portion 20.

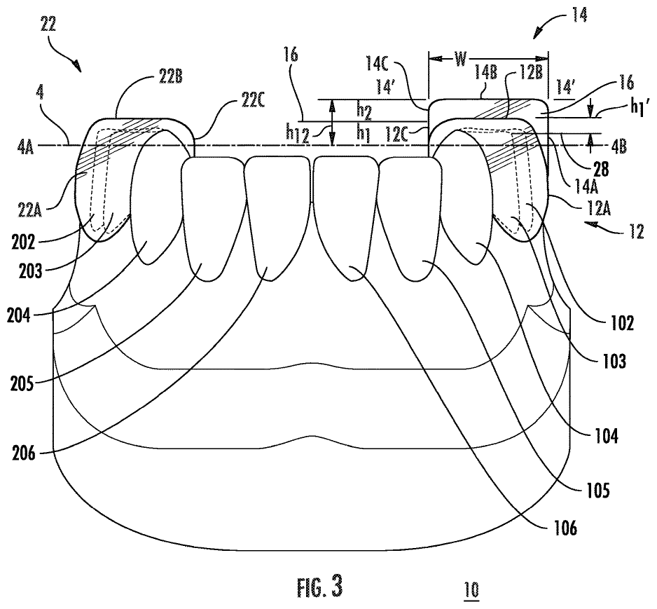

A bite block or block 14 is positioned above the right portion 12 and defines an augmented occlusal plane 14′ with respect to right occlusal plane 4B as shown in FIG. 3. The block 14 extends between at least a portion of the right occlusal plane 4B upward a distance h12=h1+h2 toward the patient's maxillary (not shown), thereby defining a portion of the augmented occlusal plane 14′, the augmented occlusal plane 14′ extending parallel to the occlusal plane 4B. The block 14 includes an outer portion 14A, an upper portion 14B and an inner portion 14C. The block 14 extends from the upper portion 12B a predetermined distance h2 toward the maxillary bone “A”. The upper surface 22B of the left portion 22 and the upper surface 12B of the right portion 12 define a plane positioned to extend a distance h1′ above an irregular surface 28 defined by teeth of a patient. In embodiments, the predetermined distance h2 which the block 14 extends toward the maxilla may be between two to seven millimeters.

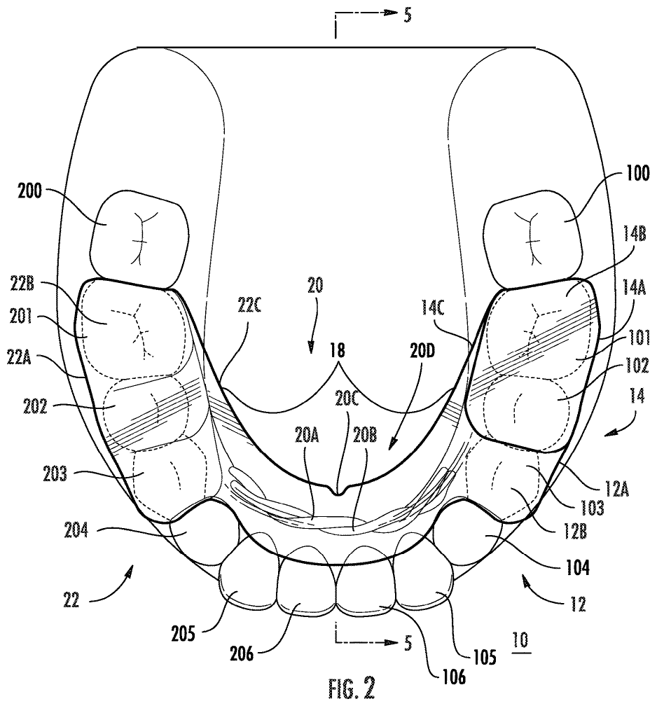

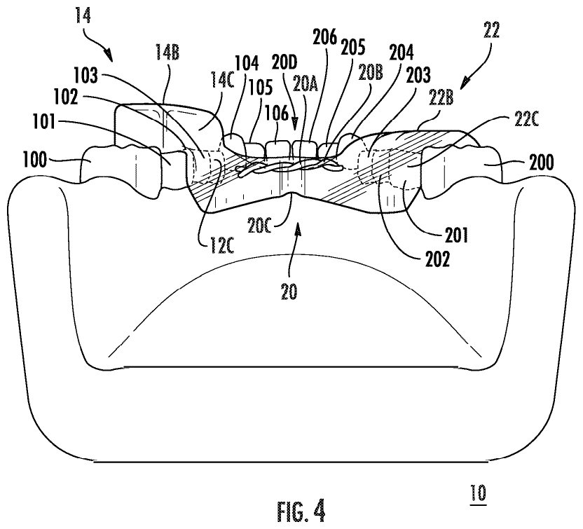

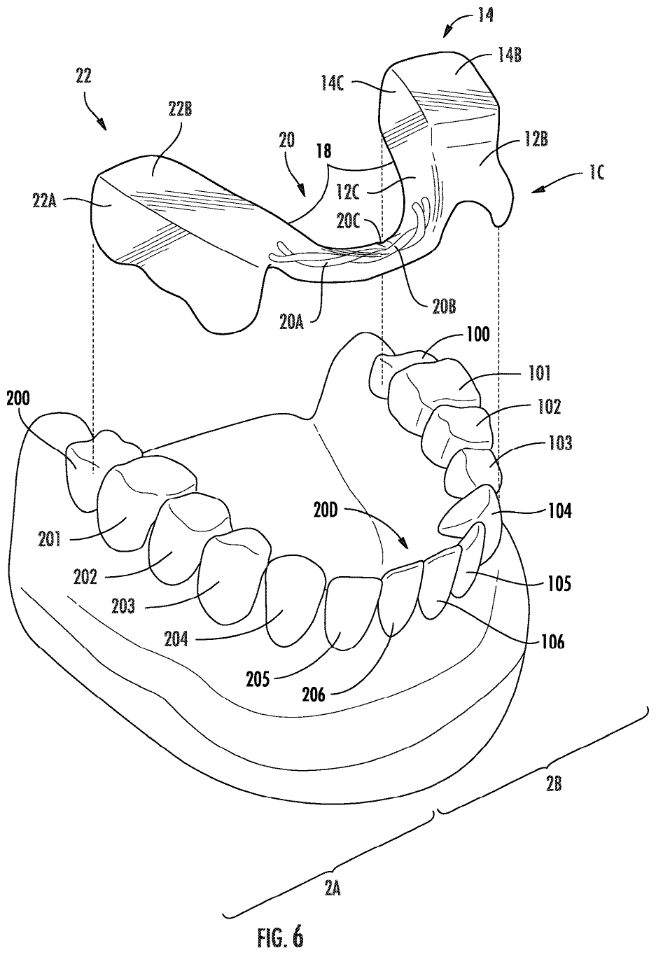

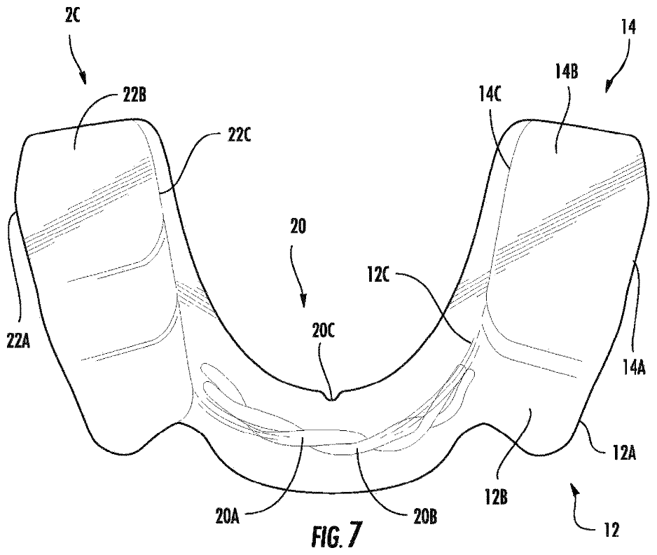

Not all patients who benefit from the orthotic device 10 will have the typical number of teeth (i.e., 32) and the orthotic 10 may be applied to situations where the patient is missing one or more teeth. It is known in the art that patients who have undergone orthodontic treatment may present with only one premolar. In addition, some patients do not have erupted third molars. Accordingly, for example, the third molar teeth are not shown in FIGS. 1-7. The upper surface 22B of the left portion 22 and the upper surface 12B of the right portion 12 defining a plane positioned to extend above irregular surface 28 defined by teeth of a patient extend at least above the region of the mandible of a patient devoted to supporting a first molar 201, 101, a second bicuspid 202, 102 and a portion of a first bicuspid 203, 103 of a patient. Thus, the bite block 14 may be disposed only along the upper surface 22B of the left portion 22 and extend above the region of the mandible of a patient devoted to supporting a first molar 201 a second bicuspid 202 and a portion of a first bicuspid 203 of a patient, whether or not the patient has each of those teeth present. In various embodiments, the bite block 14 may be disposed only along the upper surface 12B of the right portion 12 and extends above the region of the mandible of a patient devoted to supporting a first molar 101, a second bicuspid 102 and a portion of a first bicuspid 103 of a patient, whether or not the patient has each of those teeth present.

The orthotic 10 may be fabricated with a standard block 14 that is five millimeters in height h1+h1′. However, it is understood that the standard block 14 may be adjusted to a different size block 14 based on the patient's oral vertical dimensions. In various embodiments, a custom block 14 may be manufactured with more or less than a five-millimeter block. A person who has a collapsed vertical occlusal dimension from dental attrition, may utilize a seven-millimeter block to compensate for the lost vertical dimension. In various embodiments, a person with little to no occlusal vertical dimension loss may require a two-millimeter block. After fabrication, the block can be reduced with dental burs or increased with dental acrylic if necessary. It is contemplated that in various embodiments, the augmented occlusal plane may be angled such that the augmented occlusal plane tilts, or is angled, relative to the occlusal plane. An augmented angle may be necessary to accommodate the opposing dentition and can be assessed during fabrication with a model of the counter arch. The block bite plane 16 is configured to define an occlusal plane for engaging the opposing lingual and buccal cusps of the first molar and second premolar. The occlusal plane angle from mesial to distal, or from lingual to buccal, may be altered to accommodate the opposing cusps.

The upper portion 12B extends between a right first molar 101 to a portion of a right first bicuspid 103. The width of the upper portion 12B is defined by the width of the occlusal plane associated with the upper portion 12B, e.g., the right first molar 101, the right second bicuspid 102 and the right first bicuspid 103. It is contemplated that, in embodiments, the upper portion 12B may be wider or narrower than the portion of the occlusal plane 4B above the respective tooth or teeth which the upper portion 12B is positioned above.

Depending on the needs of the patient, the block 14 may be adjusted or augmented by the clinician. For example, to adjust the height during delivery and/or during treatment, the clinician may remove material from the block 14 by grinding, cutting, or otherwise removing material from the outer portion 14A, the upper portion 14B, or the inner portion 14C. In various embodiments, the clinician may add material to the outer portion 14A, the upper portion 14B, or the inner portion 14C to increase either the height h1′+h2 or width W of the block 14. Material may be added to more accurately define the augmented occlusal plane defined by the block 14, or to repair the block 14 should deformation occur while used by the patient.

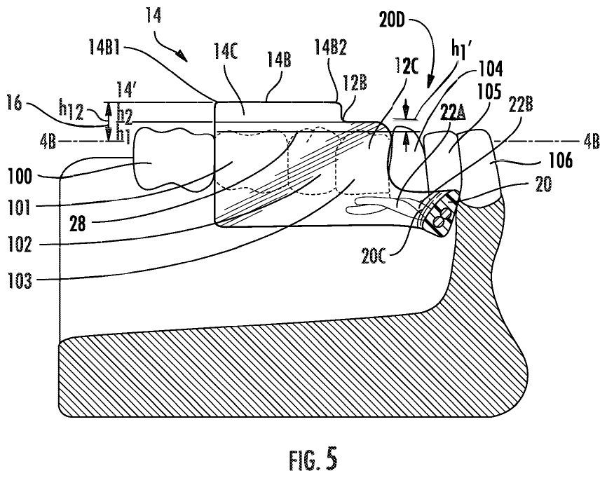

As best shown in FIGS. 1, 2, 3, 5 and 6, and most particularly in FIG. 5, the position of the upper portion 14B of the block 14 extending from position 14B1 at the rear vertical surface of first molar 101 to position 14B2 at a portion of first bicuspid 103, and thus above the first molar 101, the second bicuspid 102, and the portion of the first bicuspid 103, aligns the block 14 with a fulcrum of the maxillary suture system which is defined by motion of maxillary bone relative to the mandibular bone. As a result, in response to a patient applying force by biting on the block 14, the maximum amount of force is received by the block 14, and the maximum counter-force is received by teeth opposing the block 14, e.g., a first molar, a second bicuspid, and a portion of a first bicuspid positioned on the maxillary bone (not shown) directly above the block 14.

The left portion 22, lingual portion 20, and right portion 12 may be fabricated from dental acrylic, methyl methacrylate, pressure molded material, resin polymers, or other similar materials. Additionally, the left portion 22, lingual portion 20, and right portion 12 may be fabricated of materials capable of use in three-dimensional (3D) printing such as acrylonitrile butadiene styrene (ABS) plastic, thermoplastics such as polylactic acid, polyamide, polycarbonate, and/or other materials known in the art for use in 3D printing. In various embodiments, it is contemplated that the orthotic 10 may be fabricated with other similar compatible materials. The first and second wire 20A, 20B are fabricated of surgical steel, or other similar materials.

During manufacture, an impression is taken of the patient's teeth, along the mandible bone. The impression is then used to create a model to form the orthotic 10. Prior to hardening of the dental acrylic, the first and second wire 20A, 20B are twisted about each other into a helical shape. The first and second wire 20A, 20B are subsequently placed into the dental acrylic, and are positioned along the lingual portion 20. A clinician may add or remove material to and from the block 14 as desired during the duration of the patient's treatment.

While located in the patient's mouth and while a parafunctional occlusal force is applied (hereinafter “force”), the block 14 receives force on the upper portion 14B in response to the patient biting down, intercepting forces which would normally be received by the patient's teeth along the mandibular bone. As a result, the force is distributed from the block 14 to the teeth positioned near the block along the mandibular bone, e.g., the right first molar 101, second bicuspid 102, and the portion of the first bicuspid 103. Additionally, as the patient bites down to the block 14, a parafunctional occlusal counter-force (hereinafter “counter-force”) is applied by the block 14 to opposing teeth which come in contact with the block 14 along the maxillary bone, e.g., a right first molar, second bicuspid, and a portion of a first bicuspid positioned along the maxillary bone (not shown). As a result, force is received along the augmented occlusal plane 14′ and is focused along a subset of teeth positioned along the maxillary bone and mandible bone.

The concentration of force causes the patient to realize, either consciously or subconsciously, the application of force to the augmented occlusal plane 14′. Subsequently, the patient reduces or eliminates the application of force to the augmented occlusal plane 14′, which reduces stress applied to the patient's teeth as well as mandibular condyles. Additionally, teeth located along the mandible are prevented from coming into contact with teeth located along the maxilla, with the only occlusal contact being between the block 14 and teeth located along the maxilla opposing the block. Thus, the bite block 14 reduces the contact points between the mandibular teeth and the maxillary teeth. Further, as a result of the reduction of force applied to the mandibular condyles, the associated articular discs receive reduced forces which allow the articular discs to decompress.

As best shown in FIGS. 1, 2, 4 and 5, the lingual guide plane 20D allows the tongue (not shown) to advance to an anterior position without being blocked by the acrylic that forms the lingual arch 18 around the braided wires 20A and 20B. Consequently, the tongue moves forward and up and is removed from the throat. The periodontal ligaments of each maxillary tooth will sense reduced contact only on the bite block, therefore reducing the occlusal forces. The orthotic 10 can be fabricated in multiple ways. The traditional dental technique of acrylic (methyl methacrylate liquid and polymer) added incrementally on the custom dental model (stone and plaster) is most common. Other dental/orthodontic materials, acrylics and resin polymers may be used to fabricate the orthotic. In addition, the orthotic 10 can be fabricated via additive manufacturing (3D Printing).

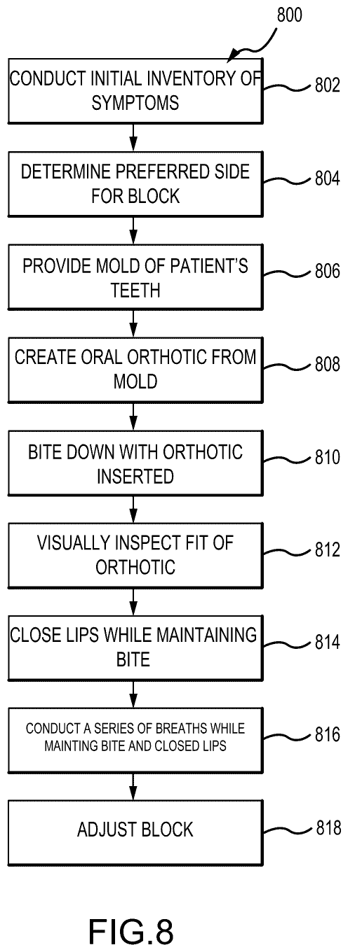

With reference to FIG. 8, a method 800 of making an oral orthotic 10 is illustrated. In various embodiments, at step 802 a clinician first determines an inventory of patient symptoms. Patient symptoms may include, but is not limited to, any of the following: HRV markers, nervous system markers, acid reflux, irritable bowel syndrome, anxiety, panic attacks, depression, ADHD, cardio-vascular disease, heart attack, stroke, high blood pressure, chronic cold hands/feet, carpel tunnel syndrome, postural hypotension, osteoporosis, ear aches, tinnitus, jaw pain, fibromyalgia, autoimmune disorders, backaches, headaches, migraines, chronic fatigue, asthma, diabetes, hypothyroidism, weight gain, obesity, clenching or grinding teeth, recurring sinus infections, chronic chapped lips, snoring, mouth breathing, frequent sleep arousals, sleep onset insomnia, asymmetrical muscle tension, or asymmetrical muscle development.

In various embodiments, at step 804 the clinician determines whether there is a preferred side for the block 14, based on the inventory of symptoms. In various embodiments, the preferred side may be associated with a best prognosis for reduction in a stress disorder or stress disorder symptoms. In various embodiments, the preferred side may be selected based on whether teeth are missing from either side, facial tension, asymmetric facial tension or drooping, or other patient symptoms.

In various embodiments, at step 806 the clinician takes a mold of the patient's teeth or mouth. In various embodiments, at step 808 clinician uses the mold to create the orthotic 10, the mold being used in conjunction with a block mold to form the block 14 along the orthotic 10. Once the orthotic 10 is created, the clinician provides the orthotic 10 to the patient, and positions the orthotic 10 in the patient's mouth. The orthotic 10 is inserted into the patient's mouth and engages one or more teeth located along the left portion 2A and the right portion 2B of the patient's mandible.

In various embodiments, at step 810 the patient bites with the orthotic 10 inserted in the patient's mouth such that one or more maxillary teeth engage the block 14. The patient need not apply an uncomfortable amount of force, merely enough to engage at least one maxillary tooth with the upper portion of the block 14.

In various embodiments, at step 812 the clinician visually inspects the fit of the orthotic 10 and looks for contacts between the block 14 and the buccal or lingual cusps of the maxillary teeth. An ideal fit includes at least two or, preferably, at least three points of contact between the block 14 and the buccal or lingual cusps of the maxillary teeth.

In various embodiments, at step 814 the patient maintains a bite and closes his/her lips around the orthotic 10. In various embodiments, during step 814 the clinician visually inspects for mentalis strain of the patient.

In various embodiments, at step 816 the patient maintains the bite with closed lips while taking a series of breaths. In various embodiments, the series of breaths may be a series of diaphragmatic breaths to engage the parasympathetic nervous system. In various embodiments, the series of breaths may be at least three breaths, at least five breaths, at least ten breaths, or any number of breaths as instructed by the clinician. In various embodiments, the clinician or patient evaluates whether the patient's tongue naturally rests out the airway, such as toward the proper tongue posture or proximate the roof of the mouth, during the series of breaths.

In various embodiments, at step 818 the clinician determines whether the block 14 must be extended or shortened based on at least one of the visual inspection of the orthotic fit, the visual inspection of mentalis strain, or evaluation of the tongue posture during the series of breaths. The orthotic 10 is removed from the patient's mouth, and the clinician adds or removes material to the orthotic 10 as desired. The clinician repeats this process until the orthotic 10 is determined to be ready by the clinician.

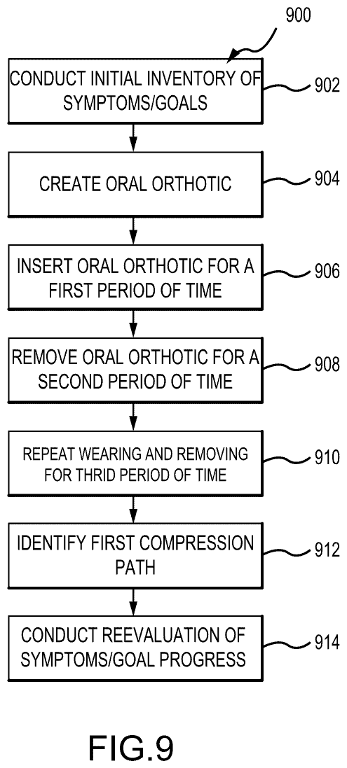

With reference to FIG. 9, a method 900 of treating a stress disorder with use of an oral orthotic 10 is illustrated. Method 900 may begin after or during various steps of method 800. In various embodiments, at step 902 a baseline is established for a patient's stress disorder symptoms and goals. In various embodiments, at step 904 an oral orthotic 10 is created for treating the patient's stress disorder symptoms and goals in accordance with method 800.

In various embodiments, at step 906 a patient wears the oral orthotic 10 for a first predetermined period of time. In various embodiments, the first predetermined period of time may be determined by a clinician in response to the inventory (or baseline) of patient symptoms and goals. The first predetermined period of time may be while the patient sleeps. The first predetermined period of time may be while the patient is awake and going about daily activities. The first predetermined period of time may be a combination of the patient's sleeping and awake time. In various embodiments, at step 908 the patient removes the oral orthotic device after the first predetermined period of time for at least a second predetermined period of time. The second predetermined period of time may be determined by the clinician based on the first predetermined period of time. In various embodiments, at step 910, the patient repeats the wearing and removing steps over a third predetermined period of time.

In various embodiments, at step 912, after the third predetermined period of time, a follow-up inventory is established for the patient's stress disorder symptoms and goals. In various embodiments, in response to steps 902-912, use of the oral orthotic results in at least one of a decrease in resting heart rate, improved HRV, improved sleep quality, reduced anxiety, reduced facial or buccolabial muscle tension, reduced bruxism, or reduced stress disorder symptoms.

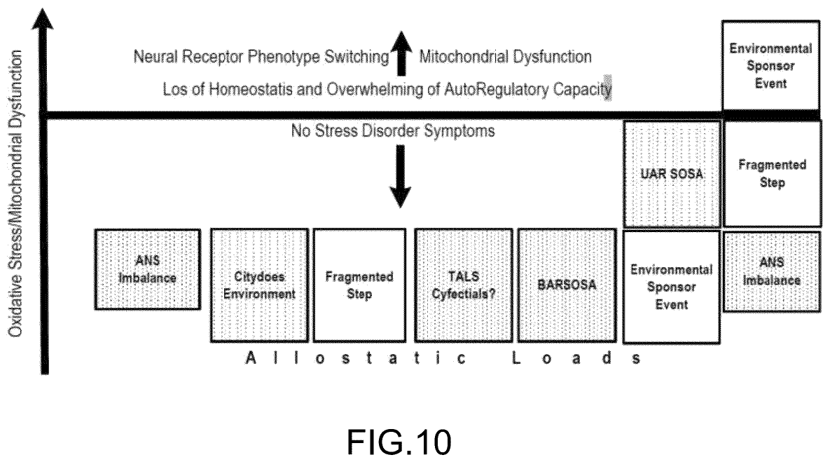

With reference to FIG. 10, a graph 1000 illustrates a relationship between bioregulatory homeostasis 1010 and auto regulatory capacity with multiple allostatic loads. While a single allostatic load 1020 may not overload the parasympathetic nervous system and a patient may not experience stress disorder symptoms, multiple allostatic loads 1020 (which each individually do not cause symptoms) may aggregate to overload the parasympathetic nervous system causing a patient to experience stress disorder symptoms. For this reason, even where a patient originally presents without certain stress disorder symptoms, use of conventional orthotics, which are associated with increased airway occlusion and interruption of diaphragmatic breathing, may create an additional stressor which itself causes loss of bioregulatory homeostasis. Further, a patient experiencing numerous allostatic loads may benefit from using the oral orthotic 10 to reduce allostatic loads merely to reduce stress disorder symptoms independent of bruxism or TMD, such as heart rate, HRV, reduced sympathetic activity, increased autoregulatory response flexibility, decreased headaches or migraines, improved biomarkers of stress, etc.

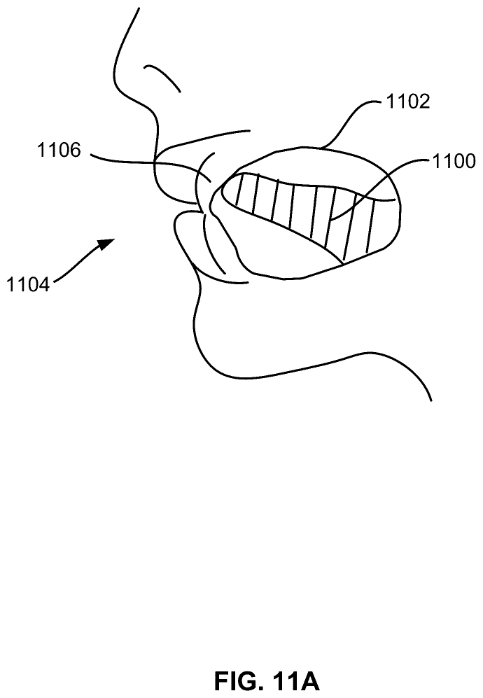

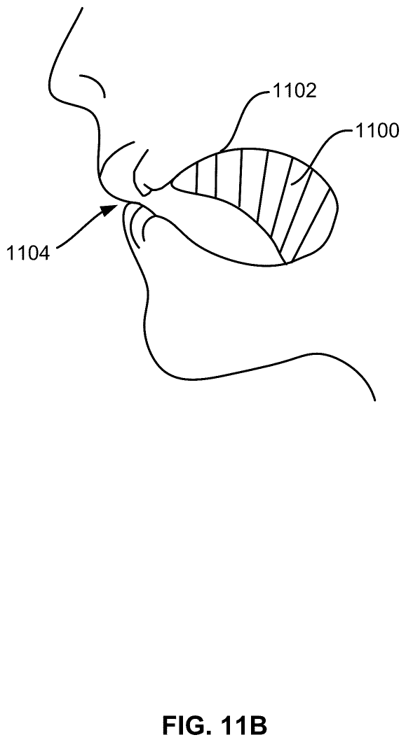

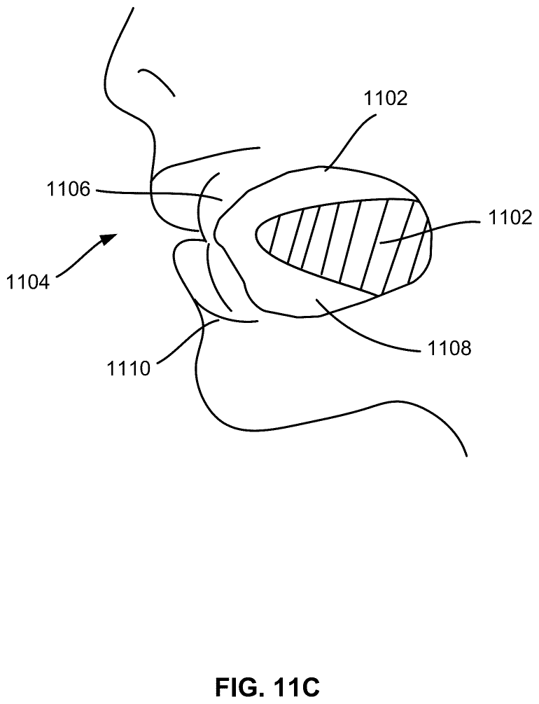

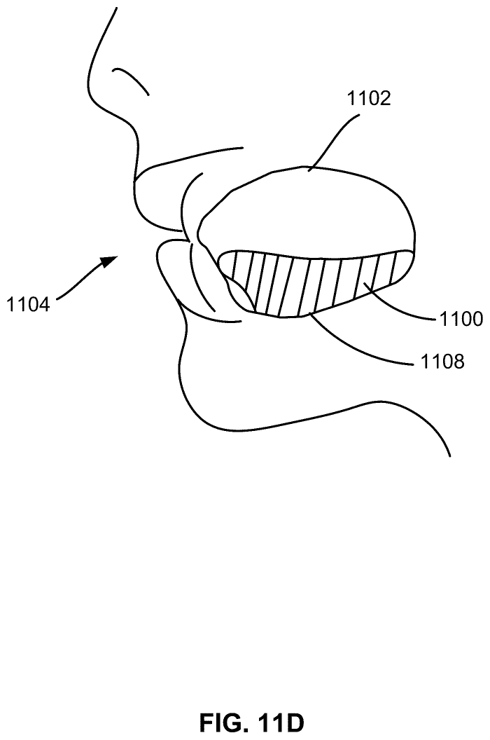

With reference to FIGS. 11A-11D, various improper and proper tongue postures are illustrated. Proper tongue posture while resting, such as when not speaking or eating or when asleep, is illustrated in FIG. 11B. In proper tongue posture, the tongue 1100 rests flat on the palate 1102 of the mouth 1104. In improper tongue posture, as illustrated in FIG. 11A, the tongue 1100 is not flat against the palate 1102 and/or a tip of the tongue is touching the maxillary teeth 1106. In improper tongue posture, as illustrated in FIG. 11C, the tongue 1100 is not flat against the palate 1102 and not touching the bottom 1108 of the mouth 1104, the maxillary teeth 1106, or the mandibular teeth 1110. In improper tongue posture, as illustrated in FIG. 11D, the tongue 1100 is flat against the bottom 1108 of the mouth 1104. In various embodiments, proper tongue posture allows for diaphragmatic and natural breath cycles without obstructions of the airpath or aggravating underlying breathing disorders. In various embodiments, use of the oral orthotic 10 triggers proper tongue posture. In various embodiments, fitting and adjusting of the oral orthotic 10 based on an evaluation of tongue posture, described below, allows for the oral orthotic 10 to treat stress disorder symptoms.

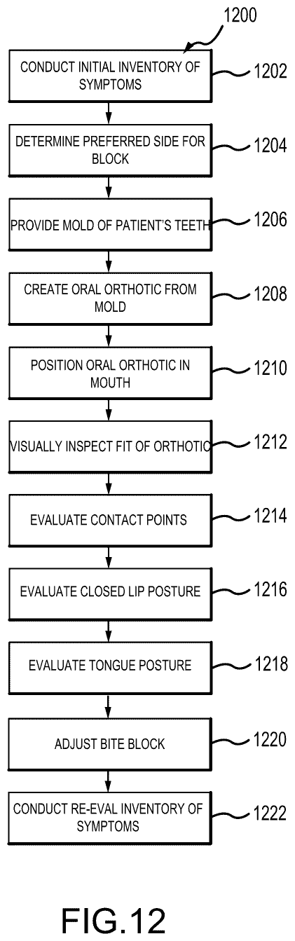

With reference to FIG. 12, a method 1200 of making an oral orthotic 10 is illustrated. In various embodiments, at step 1202 a clinician conducts an initial inventory of stress disorder symptoms. In various embodiments, at step 1204 a clinician creates a mold for the oral orthotic 10. The mold may have, in accordance with the foregoing description, at least one of a left portion, a right portion, and a lingual portion. In various embodiments, at step 1206 the clinician determines an ideal side of the mouth of the patient for a bite block 14 based on the initial inventory and the mold. The ideal side may correspond either to the left portion or the right portion of the mold, and, therefore, either to the left portion or the right portion of the oral orthotic 10. In various embodiments, at step 1208 the clinician provides an oral orthotic 10 to the patient.

In various embodiments, at step 1210 the clinician positions the oral orthotic 10 in a mouth of the patient. In various embodiments, at step 1212 the clinician visually inspects a fit of the oral orthotic 10.

In various embodiments, at step 1214 the clinician evaluates contact points of an upper portion of the bite block 14 with at least one maxillary tooth of the patient. In various embodiments, at step 1216 the clinician evaluates a closed lip posture of the patient with the oral orthotic 10.

In various embodiments, at step 1218 the clinician evaluates a tongue posture of the patient with the oral orthotic 10. In various embodiments, at step 1220 the clinician adjusts the bite block 14 as disclosed herein based on the tongue posture of the patient. In various embodiments, at step 1222 the clinician conducts a reevaluation inventory of the stress disorder symptoms. The reevaluation may be after a predetermined time. For example, the reevaluation may be after patient participates in the treatment program described in method 900. As a result of wearing the oral orthotic 10 during the treatment of the stress disorder and with the preferred tongue posture, as demonstrated by FIG. 11 and described herein, the patient may experience improved stress disorder symptoms such as improved HRV, improved heart rate, improved headache and migraine symptoms, bioregulatory homeostasis and improved nervous system response to allostatic loads (as demonstrated by FIG. 10 and described herein), anxiety relief, and stress relief.

Finally, it should be noted that while this disclosure is directed primarily to stress disorders generally, that the concepts described above can also be applied to post traumatic stress disorder, depression, anxiety, or parasympathetic nervous system disorders.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” or “at least one of A, B, and C” is used in the specification or claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching may be used throughout the figures to denote different parts but not necessarily to denote the same or different materials.

Systems, methods, and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112 (f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Finally, it should be understood that any of the above-described concepts can be used alone or in combination with any or all of the other above-described concepts. Although various embodiments have been disclosed and described, one of ordinary skill in this art would recognize that certain modifications would come within the scope of this disclosure. Accordingly, the description is not intended to be exhaustive or to limit the principles described or illustrated herein to any precise form. Many modifications and variations are possible in light of the above teaching.

1. A method for making an oral orthotic for treatment of a stress disorder, said method comprising:
conducting an initial inventory of stress disorder symptoms;
providing the oral orthotic to a patient, the orthotic having a left portion, a right portion, a lingual portion, and a bite block configured and disposed exclusively on one of the left portion or the right portion to extend over a subset of teeth of a patient along the mandibular bone of the patient;
positioning the oral orthotic in a mouth of the patient;
visually inspecting a fit of the oral orthotic;
evaluating contact points of an upper portion of the bite block with at least one maxillary tooth of the patient;
evaluating a closed lip posture of the patient with the oral orthotic; and
evaluating a tongue posture of the patient with the oral orthotic.

2. The method of claim 1, wherein the stress disorder symptoms include at least one of heart rate variability (HRV), bioregulatory homeostasis, frequency of headaches, intensity of headaches, frequency of anxiety, or intensity of anxiety.

3. The method of claim 2, further comprising determining a first period of time for wearing the oral orthotic based on the initial inventory.

4. The method of claim 3, further comprising determining a second period of time for removing the oral orthotic based on the initial inventory, and determining a third period of time for repeating the wearing and removing based on the initial inventory.

5. The method of claim 2, wherein the evaluating of the tongue posture is conducted while the patient conducts the series of breaths.

6. The method of claim 5, further comprising removing the bite block from the mouth of the patient and adjusting the bite block based on the evaluation of the tongue posture.

7. The method of claim 6, wherein the adjusting includes at least one of removing material from the bite block or adding material to the bite block.

8. The method of claim 7, further comprising visually inspecting mentalis strain of the patient during the breathing and wherein the adjusting is further based on the mentalis strain.

9. The method of claim 5, further comprising repositioning the orthotic in the mouth of the patient after the adjusting, and reevaluating the tongue posture while the patient conducts a second series of breaths.

10. A method for the treatment of a stress disorder with an oral orthotic, said method comprising:
positioning the oral orthotic in a mouth of a patient, the oral orthotic having a left portion, a right portion, a lingual portion, and a bite block configured and disposed exclusively on one of the left portion or the right portion to extend over a subset of teeth of a patient along the mandibular bone of the patient;
wearing the oral orthotic for the first predetermined period of time; and
removing the oral orthotic for a second predetermined period of time.

11. The method of claim 10, wherein the wearing includes contacting an upper portion of the bite block with at least one maxillary tooth of the patient.

12. The method of claim 11, wherein the wearing includes closing the lips of the patient around the oral orthotic to a closed lip posture.

13. The method of claim 12, wherein the wearing includes breathing a series of breaths while maintaining the contact and the closed lip posture.

14. The method of claim 13, further comprising, in response to the wearing the oral orthotic, maintaining a proper tongue posture during the breathing.

15. The method of claim 14, further comprising repeating the wearing and removing steps over a third predetermined period of time, wherein the first period of time, the second period of time, and the third period of time are based on at least one patient-specific symptom of the stress disorder.

16. A method for making an oral orthotic for treatment of a stress disorder, said method comprising:
conducting an initial inventory of stress disorder symptoms;
creating a mold for the oral orthotic, the mold having a left portion, a right portion, and a lingual portion;
determining an ideal side of the mouth of the patient for a bite block based on the initial inventory and the mold;
providing the oral orthotic to a patient, the oral orthotic having a bite block configured and disposed exclusively on the ideal side to extend over a subset of teeth of a patient along the mandibular bone of the patient;
positioning the oral orthotic in a mouth of the patient; and
visually inspecting a fit of the oral orthotic.

17. The method of claim 16, wherein the ideal side corresponds either to the left portion or the right portion.

18. The method of claim 17, further comprising:
evaluating contact points of an upper portion of the bite block with at least one maxillary tooth of the patient;
evaluating a closed lip posture of the patient with the oral orthotic; and
evaluating a tongue posture of the patient with the oral orthotic.

19. The method of claim 18, further comprising adjusting the bite block based on the tongue posture of the patient.

20. The method of claim 19, further comprising conducting a reevaluation inventory of the stress disorder symptoms.
